(19) United States
(12) Patent Application Publication
(10) Pub. No.: US 2019/0313998 A1
(43) Pub. Date: Oct. 17, 2019

(54) Systems and Methods for Facilitating Auscultation Detection of Vascular Conditions

(71) Applicant: NEPHTECH PTE LTD, Singapore (SS)

(72) Inventors: Soo Ghim LIM, Singapore (SG); Yanling TOH, Singapore (SG)

(21) Appl. No.: 16/474,561

(22) PCT Filed: Dec. 29, 2017
(86) PCT No.: PCT/SG2017/050656

§ 371 Date: Jun. 28, 2019

(30) Foreign Application Priority Data
Dec. 29, 2016 (SG) 10201610939W

(51) Int. Cl. A61B 7/02; A61B 5/02; A61B 5/00

(52) U.S. Cl. CPC A61B 7/026; A61B 5/02007; A61B 5/021; A61B 5/489; A61B 5/6843

(57) ABSTRACT

Systems and methods for facilitating auscultation detection of vascular conditions. A detector module for facilitating auscultation detection of vascular conditions, comprising: one or more auscultation detectors, each of the one or more auscultation detectors configured to acquire auscultation signals associated with at least one blood vessel; a securing means configured to secure the detector module superficially onto a user's skin such that the auscultation signals associated with the at least one blood vessel can be acquired; and a microprocessor module that is in communication with the one or more auscultation detectors and is configured to transmit the acquired auscultation signals to an external module that is in communication with the detector module.

### FIELD OF INVENTION

The present invention relates broadly, but not exclusively, to systems and methods for facilitating auscultation detection of vascular conditions.

### BACKGROUND

Auscultation has been the primary mode of capturing and analysis of internal sounds from the body. It is performed for the purposes of examining the circulatory system, respiratory system and even the gastrointestinal system. It is a critical part of physical examination of a patient and is routinely used to provide strong evidence for pathological clinical conditions.

To identify abnormalities such as narrowing or stenosis in the circulatory system, a bruit is an audible sound typically associated with turbulent blood flow that clinicians seek out. This is particularly pertinent to seek out bruits in the body, especially within large superficial vasculature, such as within the heart—cardiac valvular murmurs radiating to the neck, cervical arteries (carotid artery bruits), cervical veins (cervical venous hum), and/or arteriovenous (AV) connections. If narrowing becomes extensive in such vasculature, adequate blood flow may not be possible past the point of stenosis and thus may result in injuries to tissues distal to the narrowed lumen.

For example, a narrowing to the coronary vessels providing blood to the heart can lead to cardiovascular dysfunction and decrease blood flow, leading to a heart attack.

Strokes can either result from blockage of blood flow in the cerebral vessels due to constriction of the vessel, or from carotid artery narrowing from the buildup of plaque (fibrous and fatty deposits) within the lumen of arteries. The latter causes many incidences of stroke cases. This may also be discovered by auscultation of the carotid artery on the neck region.

Aside from physical examination, other tests for confirmation could include Doppler carotid ultrasound, carotid angiography, magnetic resonance angiography or computed tomography, which are all technologies found within a hospital only.

Getting treatment early upon an onset of stroke and full recovery could be expected if a blocked artery is treated within four hours of symptom onset. If not treated early, this can cause lasting brain damage and long term disability such as vision, speech and paralysis problems. It is paramount to diagnosing such blockages early to reduce the onset of other complications.

In another disease condition, Chronic Kidney Disease (CKD) is a kidney disease condition that describes and classifies declining kidney functions and performance. CKD is typically classified into five stages with patients classified with CKD stage 5 when their kidney function drops below 10% of normal functions. This stage is also known as End Stage Renal Disease (ESRD). In ESRD, the body is unable to normally and effectively remove bodily waste resulting in toxin build-up in the body. If left untreated, toxin accumulation could lead to various side effects like fatigue, nausea and ultimately death. Currently there are only two treatments for ESRD—Kidney transplant or Dialysis treatment. With the shortage of kidney donors and long waiting lists, a majority of ESRD patients are on dialysis treatment, i.e. artificial means of removing bodily waste and toxins. There are two types of dialysis treatments, hemodialysis (blood dialysis) and peritoneal dialysis (water dialysis) out of which more than 89% of global ESRD patients are on hemodialysis.

To enable commencement of hemodialysis treatment, a suitable dialysis blood vessel has to be specially created by a vascular surgeon. This dialysis blood vessel, known as Arteriovenous Fistula (AVF) or Arteriovenous Graft (AVG), is typically created through anastomosis surgery. These AVFs or AVGs become vascular accesses or point of access (needling) during dialysis treatment for the removal of bodily waste and toxins.

Through frequent needling of these AVFs and AVGs (typically thrice weekly for the rest of a patient's life), the vascular access is subjected to adverse development of complications such as stenosis (narrowing of blood vessel), thrombosis (blood clot resulting in vascular blockage), hematoma (unable to achieve blood clot at needle puncture site leading to excessive blood), aneurysm (weakening of section of blood vessel resulting in abnormal localised changes in blood vessel lumen, typically translating into bumps in the blood vessel), etc. Stenosis accounts for the highest incidence of complications and can lead to formation of thrombosis. As such, it is critical for vascular conditions to be regularly monitored for a presence of complications.

Currently, there are several commercial techniques to calculate changes to blood flow rate or static/dynamic pressure changes. These current techniques are not without its shortcomings, including operator skill dependency, high capital costs and/or lengthy assessment duration.

One commercial technique is auscultation, which is available to treatment centres such as hospital dialysis units and dialysis centres by renal nurses. Auscultation primarily involves the use of ultrasound and pressure sensing technology. Examples of currently available solutions include Transonic® (Ultrasound-based), HemaMetrics (Ultrasound), Vasc-alert (pressure) and Fresenius In-line Dialysance (pressure). Transonic® utilises an ultra-dilution principle involving a lengthy assessment process to measure the in-situ vascular access blood flow. This technique can only be performed during hemodialysis treatment. Both the Vasc-alert and Fresenius In-line Dialysance assessment techniques are based on changes in intra-access dynamic pressure during dialysis treatment. The use of static pressure as markers has been demonstrated, especially for AVG, to be inaccurate. Dynamic pressure, on the other hand, has been demonstrated to have good vascular condition prediction and reliability. However, their use is limited to during dialysis treatment and usually has to be performed throughout the prescribed dialysis treatment duration. In summary, current auscultation and pressure sensing techniques are restricted to use during dialysis treatment, hence can only be performed in hospital treatment units or dialysis centres.

Using ultrasound technology, a signal can be converted into ultrasound imaging for visualisation of vascular conditions. Common ultrasound imaging techniques for vascular accesses include Duplex Doppler Ultrasound, Angiography, and Variable Flow Doppler Ultrasound. Such advanced imaging techniques require specially trained operators and are typically only available for in-hospital use. The need for highly trained operators (radiographers or trained clinicians) translates to high cost, and hence not usually suitable for regular prophylactic monitoring of the vascular access. Such advanced techniques are usually used on an irregular, on-demand basis or to verify the presence and location of a complication for interventional action.

Another commercial technique involves biochemical alterations. Techniques that use biochemical markers include Glucose pump infusion, urea dilution, and differential conductivity (GAMBRO). These techniques typically employ an indirect method of assessing changes in respective biomarkers and correlating with vascular conditions. The main drawback is inconsistent or unreliable assessment outcomes.

A need therefore exists to provide systems and methods for facilitating auscultation detection of vascular conditions that seek to address at least some of the above problems.

### SUMMARY

According to a first aspect, there is provided a detector module for facilitating auscultation detection of vascular conditions, comprising: one or more auscultation detectors, each of the one or more auscultation detectors configured to acquire auscultation signals associated with at least one blood vessel; a securing means configured to secure the detector module superficially onto a user's skin such that the auscultation signals associated with the at least one blood vessel can be acquired; and a microprocessor module that is in communication with the one or more auscultation detectors. The microprocessor module is configured to: (i) receive the acquired auscultation signals from the one or more auscultation detectors and (ii) transmit the acquired auscultation signals to an external module that is in communication with the detector module.

The detector module may comprise a plurality of the auscultation detectors, wherein the microprocessor module is further configured to: receive, from each of the plurality of auscultation detectors, respective acquired auscultation signals; and determine, based on one or more pre-defined parameters, which one or ones of the acquired auscultation signals to transmit to the external module.

The one or more pre-defined parameters may comprise: detected pressure of the auscultation detector(s) against the user's skin, signal-to-noise ratio, dynamic range of the auscultation detector(s), frequency response of the auscultation detector(s) and/or auscultation signal strength. The acquired auscultation signal with a highest auscultation signal strength may be transmitted to the external module.

Each of the one or more auscultation detectors may comprise an actuating mechanism that is configured to move the auscultation detector along a z-axis, either away or towards the user's skin.

The microprocessor module may be further configured to: determine a distance between the auscultation detector(s) and (i) the user's skin or (ii) the at least one blood vessel; and provide a feedback to the user that is indicative of the distance between the auscultation detector(s) and (i) the user's skin or (ii) the at least one blood vessel.

The microprocessor module may be further configured to process the acquired auscultation signals to generate corresponding blood flow characteristic data of the at least one blood vessel, wherein the blood flow characteristic data comprises acoustic signals indicative of vascular narrowing.

The detector module may further comprise a memory module having stored therein an artefact library. The microprocessor module may be further configured to reference the artefact library to determine a presence of artefacts in the auscultation signals associated with the at least one blood vessel acquired by the one or more auscultation detectors.

According to a second aspect, there is provided a method for facilitating auscultation detection of vascular conditions, comprising: providing a detector module comprising one or more auscultation detectors, each of the one or more auscultation detectors configured to acquire auscultation signals associated with at least one blood vessel; providing a securing means configured to secure the detector module superficially onto a user's skin such that the auscultation signals associated with the at least one blood vessel can be acquired; and providing a microprocessor module that is in communication with the detector module. The microprocessor module is configured to: (i) receive the acquired auscultation signals from the one or more auscultation detectors and (ii) transmit the acquired auscultation signals to an external module that is in communication with the detector module.

The method may further comprise: providing a plurality of the auscultation detectors; and configuring the microprocessor module to: receive, from each of the plurality of auscultation detectors, respective acquired auscultation signals; and determine, based on one or more pre-defined parameters, which one or ones of the acquired auscultation signals to transmit to the external module.

The one or more pre-defined parameters may comprise: detected pressure of the auscultation detector(s) against the user's skin, signal-to-noise ratio, dynamic range of the auscultation detector(s), frequency response of the auscultation detector(s) and/or auscultation signal strength. The acquired auscultation signal with a highest auscultation signal strength may be transmitted to the external module.

The method may further comprise: providing each of the one or more auscultation detectors with an actuating mechanism that is configured to move the auscultation detector along a z-axis, either away or towards the user's skin.

The method may further comprise: configuring the microprocessor module to: determine a distance between the auscultation detector(s) and (i) the user's skin or (ii) the at least one blood vessel; and provide a feedback to the user that is indicative of the distance between the auscultation detector(s) and (i) the user's skin or (ii) the at least one blood vessel.

The method may further comprise: configuring the microprocessor module to: process the acquired auscultation signals to generate corresponding blood flow characteristic data of the at least one blood vessel, wherein the blood flow characteristic data comprises acoustic signals indicative of vascular narrowing.

The method may further comprise: providing a memory module having stored therein an artefact library, the memory module being in communication with the microprocessor module; and configuring the microprocessor module to: reference the artefact library to determine a presence of artefacts in the auscultation signals associated with the at least one blood vessel acquired by the one or more auscultation detectors.

### BRIEF DESCRIPTION OF THE DRAWINGS

Embodiments and implementations are provided by way of example only, and will be better understood and readily apparent to one of ordinary skill in the art from the following written description, read in conjunction with the drawings, in which:

FIG. 1 shows a schematic diagram of a system for facilitating auscultation detection of vascular conditions, according to an example embodiment.

FIGS. 2(a), (b) and (c) show schematic diagrams of a detector module with a strap, according to an example embodiment.

FIG. 2(d) shows a schematic diagram illustrating an arrangement of detectors in a 2D array, according to an example embodiment.

FIG. 3 shows a schematic diagram of a detector module comprising a sleeve, according to an example embodiment.

FIGS. 4(a), (b)(i) and (b)(ii) show schematic diagrams of a detector module comprising a cuff, according to example embodiments.

FIGS. 5(a), (b) and (c) show schematic diagrams of z-axis adjustment mechanisms of a detector module, according to example embodiments.

FIGS. 6(a) and (b) show user interfaces displayed on a user module, according to an example embodiment.

FIG. 7 shows a data flow diagram illustrating a method for facilitating auscultation detection of vascular conditions, according to an example embodiment.

FIG. 8 shows a schematic diagram of a computer system suitable for use in executing at least some steps of the method for facilitating auscultation detection of vascular conditions and/or for realizing at least a part of the system for facilitating auscultation detection of vascular conditions.

### DETAILED DESCRIPTION

Embodiments will be described, by way of example only, with reference to the drawings. Like reference numerals and characters in the drawings refer to like elements or equivalents.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “receiving”, “scanning”, “calculating”, “determining”, “replacing”, “generating”, “initializing”, “outputting”, or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a computer suitable for executing the various methods/processes described herein will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM mobile telephone system. The computer program when loaded and executed on such a computer effectively results in an apparatus that implements the steps of the preferred method.

Embodiments of the invention relate to systems and methods for facilitating auscultation detection of vascular conditions. One exemplary embodiment uses auscultation techniques to detect changes in blood flow of a blood vessel (e.g. a vascular access) for the assessment of vascular conditions. Vascular conditions and complications include, but are not limited to: stenosis (narrowing of blood vessel), thrombosis (blood clot resulting in vascular blockage), hematoma (unable to achieve blood clot at needle puncture site leading to excessive blood), and aneurysm (weakening of section of blood vessel resulting in abnormal localised changes in blood vessel lumen, typically translating into bumps in the blood vessel).

Embodiments using auscultation techniques can provide a sensitive and reliable prediction for a presence and degree of stenosis by employing indirect methods derived from blood flow characteristics. Specifically, the blood flow characteristics are processed and analyzed for identification and development of stenosis to aid the assessment of vascular conditions.

In one embodiment, there is provided a system to measure and monitor vascular conditions of patients, in particular in large superficial vasculature such as, but not limited to, the Arteriovenous Fistula (AVF) or Arteriovenous Graft (AVG) vascular accesses, carotid artery, etc. In the case of the vascular access, embodiments involve a non-invasive technique (i.e. does not require needles to be inserted into a hemodialysis patient's vascular access before assessment can be performed) and does not require continuous assessment throughout dialysis treatment. This non-invasive technique advantageously allows for assessment to be performed outside of the dialysis treatment window, and can be performed as frequently as necessary for prophylactic and long term monitoring.

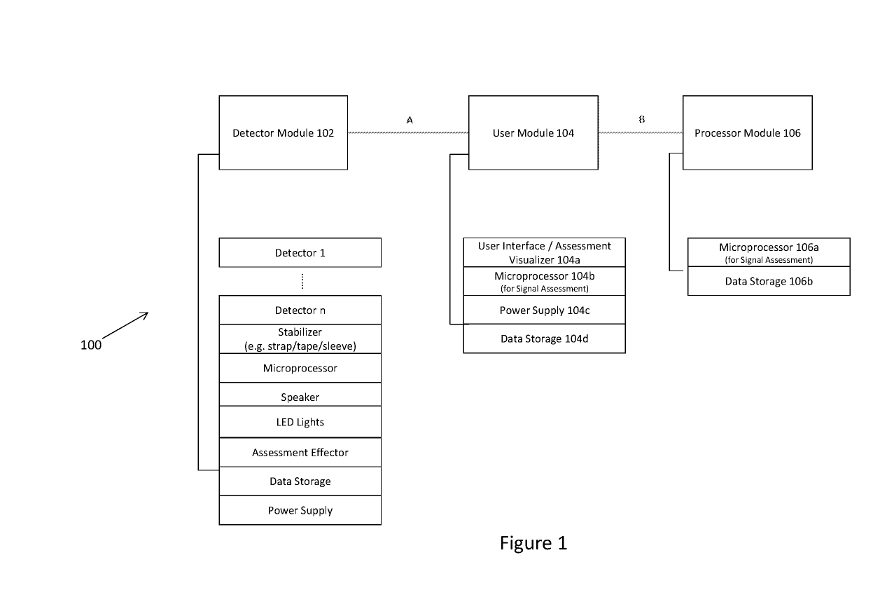

FIG. 1 shows a schematic diagram of a system for facilitating auscultation detection of vascular conditions, according to an example embodiment. The system 100 is a vascular assessment system that includes (i) a detector module 102 that is configured to acquire physiological signals from one or more vasculature, (ii) a user module 104 that is configured to perform signal assessment and provide a user interface, and (iii) a processor module 106 that is configured to perform further signal assessment and provide additional data processing and storage.

The detector module 102 is in communication with the user module 104 via communication modality A, e.g. wired, USB, Bluetooth and Wi-Fi. The user module 104 is in communication with the processor module 106 via communication modality B, e.g. Wi-Fi, 3G, 4G and 5G networks.

Although only one detector module 102, user module 104, and processor module 106 are shown in FIG. 1, the system 100 may include more than one detector module, user module, and/or processor module. For example, a plurality of user modules may be in communication with a processor module at any one time.

The detector module 102 is configured to collect/acquire physiological signals emitted from one or more vasculature. The detector module 102 may be implemented as an individual portable unit that can be handheld and include mechanical components, optical imaging modules, and other auscultation detectors/sensors, used in any permutation.

The collected auscultation signals can be processed by the user module 104 and/or the processor module 106 to perform a variety of functions such as, but not limited to, (a) flow characteristic computation, (b) verification of flow characteristics, and/or (c) recording of background signal for de-noising purposes.

The detector module 102 can comprise a plurality of auscultation detectors (Detector 1 . . . Detector n) with the plurality of detectors arranged in a linear array along an intended vasculature. Each auscultation detector (Detector 1 . . . Detector n) may function as an individual unit and the distance between each auscultation detector can vary based on a user's vasculature. The distance between each auscultation detector can be determined using a distance measurement module built into the detector module 102, such as, but not limited to, mechanical ruler measurement, laser distance sensing, Bluetooth triangulation, electrical impedance means of measuring distance, or a combination thereof. In this manner, embodiments allow customisation to individual patient's vasculature at various distances from the intended blood vessel. Specifically, distances between detectors for different users are recorded in order to optimize positioning over a variable anatomy. Distance can be determined based on a selection of highest signal strength to cater to different users' anatomy.

As each vasculature is uniquely developed due to anatomical variances among users, embodiments seek to provide a system that can address a wide range of vasculature for repeatability and reproducibility. The positioning of the detector module 102 may be based on pre-determined location landmarks such as proximity to an anastomotic junction, surgical scars on a user's body, specific distance from palm base or optical imaging of sub-skin vasculature as landmarks.

As such, the detector module 102 includes means to aid better positioning, means to optimize signal acquisition; and means for system level signal identification and verification.

### Improved Positioning

The detector module may 102 comprise one or more auscultation detectors (Detector 1 . . . Detector n), electronics and circuitry wirings. Each of the plurality of auscultation detectors can comprise one or more of: a contact pressure sensor, a piezo sensor, a microphone, and other types of auscultation detector/sensor, used in any permutation (e.g. all contact pressure sensors, two contact pressure sensors and one microphone, etc). A cushioning material such as, but not limited to, hydrogel or foam may be placed at the base of the detector module 102 for user comfort.

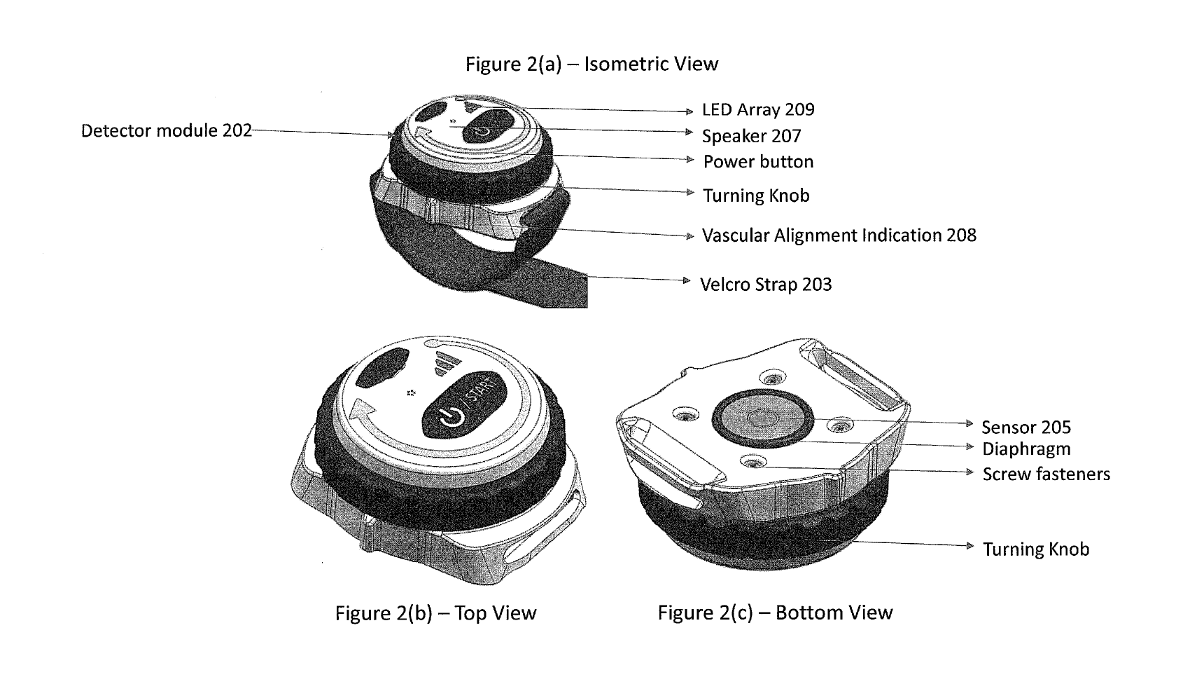

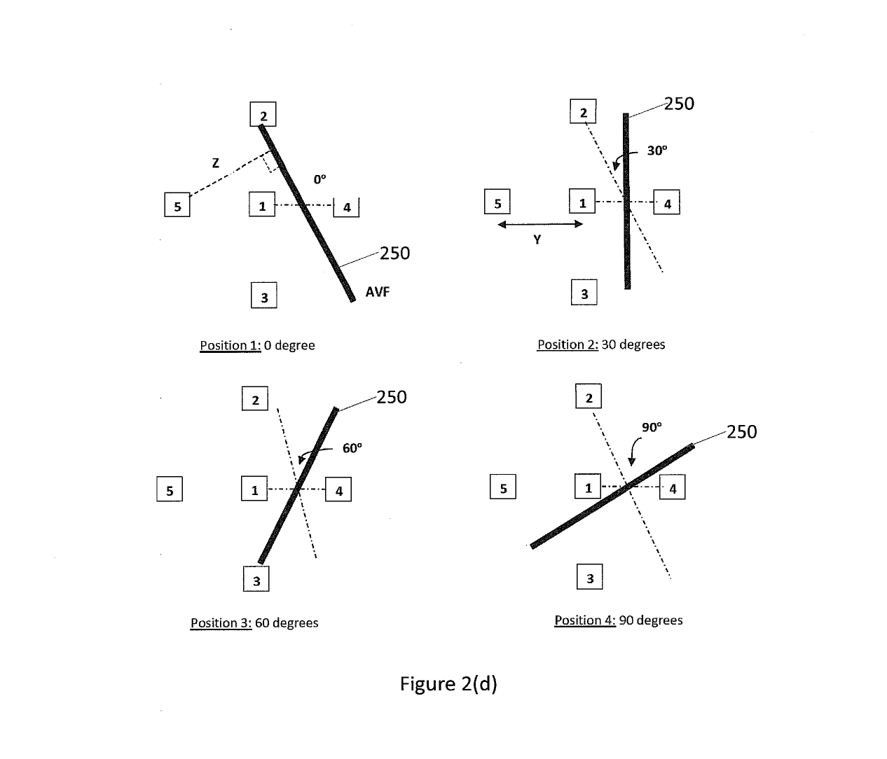

FIGS. 2(a), (b) and (c) show schematic diagrams of a detector module 202 with a strap 203, according to an example embodiment. FIG. 2(a) shows an isometric view of the detector module 202, FIG. 2(b) shows a top view of the detector module 202, and FIG. 2(c) shows a bottom view of the detector module 202. To achieve consistent pressure and measurement stability on a vasculature while taking measurement, one or more auscultation sensors 205 are radially fastened around a patient's arm for consistency of readings through the strap 203. The strap 203 may be of variable dimensions/sizes catered for various patient populations, and includes means of tightening and fastening the strap in position, e.g. Velcro, buckles, buttons etc.

In another implementation, a detector module comprises a 2D array of auscultation detectors. With a 2D array of auscultation detectors, accurate positioning of the detector module over a vasculature is relatively less critical. The array of detectors can determine which detector, in plurality, is directly over the intended vasculature based on a single or set of pre-defined parameters such as, but not limited to, detected pressure of the auscultation detector(s) against the user's skin, signal-to-noise ratio, dynamic range of the detector, frequency response of the detector and/or auscultation signal strength. This process can be performed algorithmically without a need for any user action.

FIG. 2(d) shows a schematic diagram illustrating an arrangement of detectors in a 2D array, according to an example embodiment. The array comprises five auscultation detectors, Detector 1, 2, 3, 4 and 5, arranged in a cross-shaped configuration. The array of auscultation detectors may be placed over a vasculature 250. The arrangement of each detector in the array can be varied based on characteristics of the vasculature 250 (e.g. angles of the vasculature 250). In particular, the inter-detector distance, y, and the perpendicular distance to the vasculature 250, z, can be varied depending on the characteristics of the vasculature 250.

The collection of signals from the multiple detectors in a 2D array may be based on factors including, but not limited to, a particular detector's proximity to the intended vasculature 250; and a received signal strength from the vasculature 250, where the individually received signals are independently detected and compared using a microprocessor. The signal(s) from the most optimal position are selected for processing and assessment of vasculature conditions. For example, with reference to FIG. 2(d), in Positions 1 and 3, there is one detector directly above the vasculature 250. In Positions 2 and 4, the vasculature 250 is at a distance away from any of the detectors in the array. The received signal strengths from Detectors 1, 2, 3, 4 and 5 are collected and compared by the microprocessor. In an example scenario, in Position 1, the received signal strength from Detector 2 is determined to be the highest relative to the received signal strengths from Detectors 1, 3, 4 and 5. Accordingly, the received signal strength from Detector 2 is selected for processing and assessment of vasculature conditions. In another example scenario, in Position 2, the received signal strength from Detector 1 is determined to be the highest relative to the received signal strengths from Detectors 2, 3, 4 and 5. Accordingly, the received signal strength from Detector 1 is selected for processing and assessment of vasculature conditions.

The consideration of the arrangement, configuration, number and type of detectors to be used may be based on information to be collected. This information may be related to electrical, biochemical, mechanical aspects can be separately collected and/or compared for purposes such as signal verification, clinical indication correlation for diagnostics.

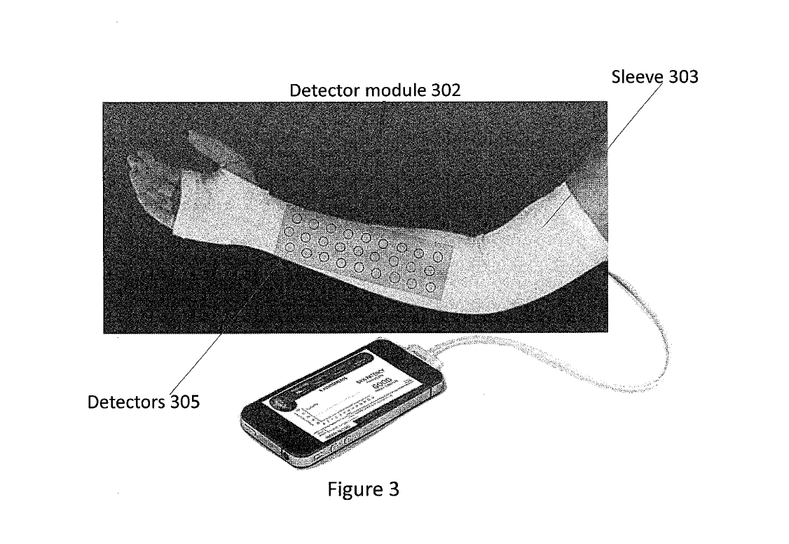

FIG. 3 shows a schematic diagram of a detector module 302 comprising a sleeve 303, according to an example embodiment. The sleeve 303 is preferably in the form of a breathable flexible sleeve that a patient can attach around his/her arm over the vasculature. The sleeve material can include, but is not limited to, fibre-based materials and polymers such as rubber to provide flexibility over the vasculature. A 2D array of detectors 305 is disposed on an inner surface of the sleeve to collect signals from the vasculature, hence exerting minimal or constant pressure on the vasculature while optimizing physical skin contact to ensure optimal detector readings, regardless of the overall contour of body for its placement.

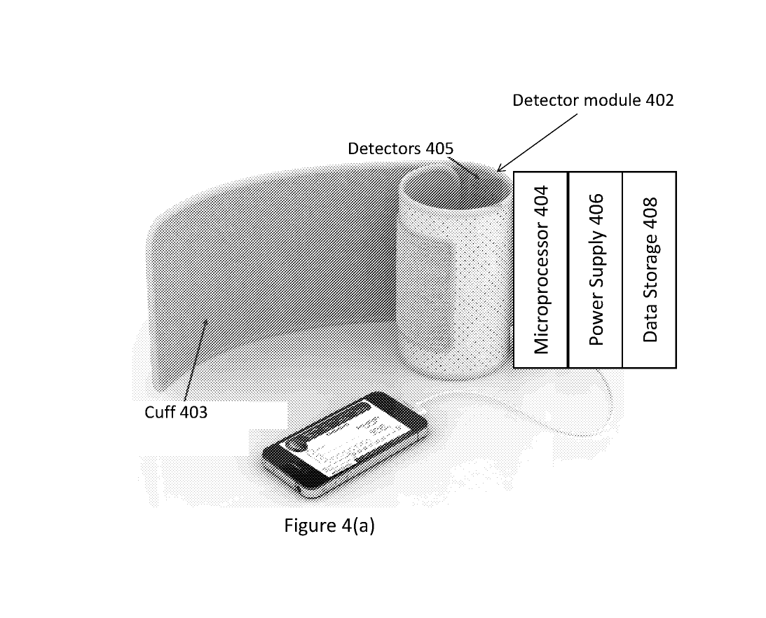

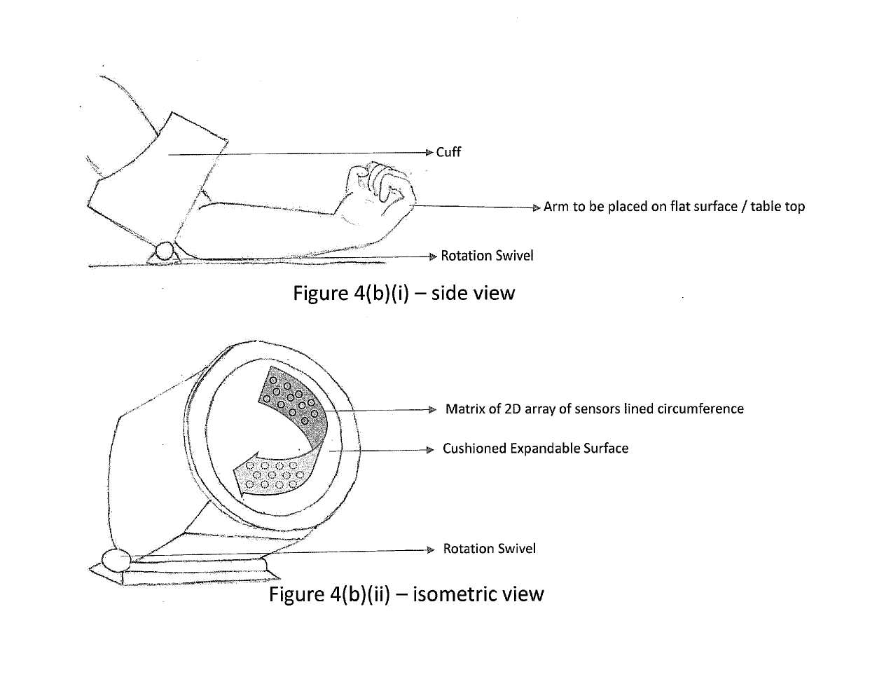

FIGS. 4(a) and (b)(i)/(ii) show schematic diagrams of a detector module 402 comprising a cuff, according to example embodiments. As shown in FIG. 4(a), a detector module 402 can include a cuff 403 that can be strapped around a vasculature, with a 2D array of detectors 405 disposed on an inner side of the cuff 403 for contact with the patient's skin. The cuff 403 can be in various configurations, such as flat plates that rest on the arm or rounded curved casts made of a stiff and rigid material. The cuff 403 can include means to enhance detector and skin contact, e.g. cuffs that are designed to be inflatable, through means not limited to air, water and gel, etc. The 2D array of detectors 405 is disposed on an underside of the plates to achieve maximum contact. The detector module 402 may further comprise a microprocessor 404, a power supply module 406 and a data storage module 408. FIGS. 4(b)(i) and (ii) show a station cuff variant (side view and isometric view, respectively) and may have similar components as the detector module 402 shown in FIG. 4(a).

The detector module 302/402 with a sleeve or cuff comprising a 2D array of detectors can algorithmically determine (based on pre-defined parameters) which detector(s) is nearest to the vasculature and initiate auscultation signal(s) collection, processing and assessment of vascular condition without any user action.

### Optimizing Signals for Collection

Z-Axis Positioning

The X-Y axis positioning of a detector module can be performed manually by end-users over an intended vasculature. This can also be guided by optical imaging capability of the detector module that helps to illuminate and identify sub-skin vasculature for more accurate positioning of the detector module over the intended vasculature. Following the placement of the detector module along the intended vasculature, the detector module is moved into position (over the vasculature) based on adjustment along the z-axis (i.e. perpendicular to the skin) in order to enhance and refine the signals received from the vasculature. Such detector adjustment threshold can be based on, but not limited to, predetermined pressure strength or detected signal strength.

In other words, positioning of a detector module (or auscultation detectors) over the vasculature (x-axis & y-axis) and vertical control of the detector module (or auscultation detectors) (z-axis) can be based on predetermined parameters such as pressure strength and/or detected signal strength. The distance of perpendicular protrusion (i.e. in the z-axis) of the detector module (or auscultation detectors) effected can be based on several considerations, including but not limited to: the depth of the blood vessel below the skin surface (based on population studies and/or photo-acoustics detection such as ultrasound verification for the particular vasculature), and pressure exerted by the detector module before it impinges on the flow dynamics of the blood vessel.

The movement of the detector module can be specifically configured to facilitate a one-handed operation. It may be based on, but not limited to: (i) actuated movement (for the cuff as described above with reference to FIGS. 4(a) and (b)(i)/(ii)); (ii) mechanical movement; and/or (iii) physical tightening of the strap as described above with reference to FIGS. 2(a), (b) and (c).

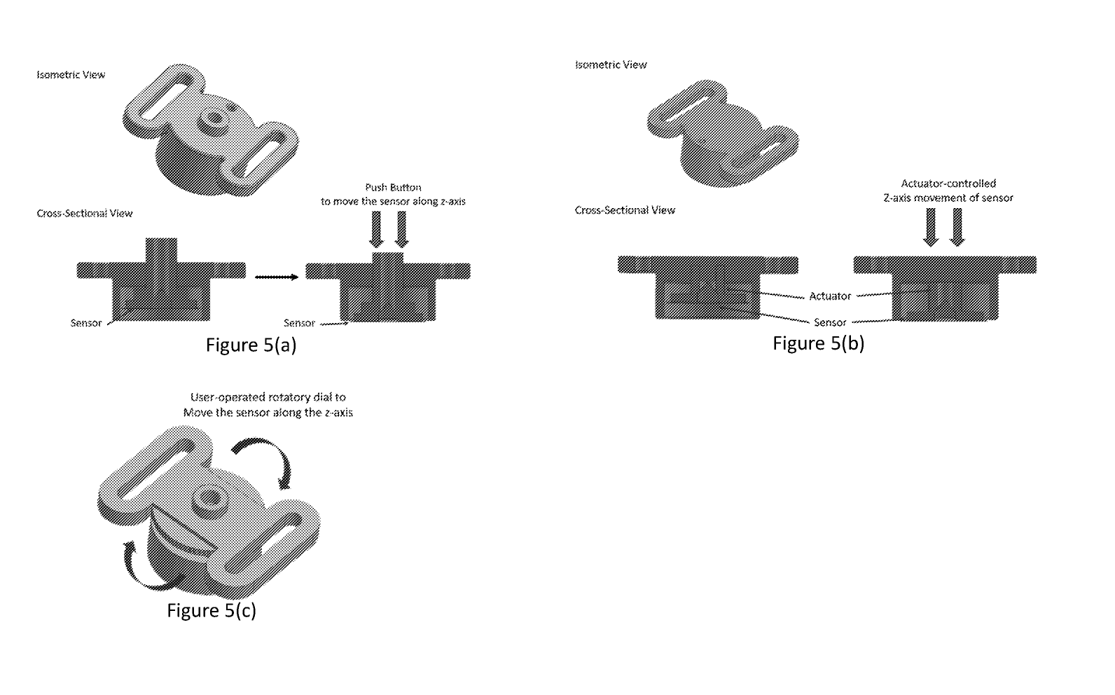

FIGS. 5(a), (b) and (c) show schematic diagrams of z-axis adjustment mechanisms of a detector module, according to example embodiments. FIG. 5(a) shows a push button mechanism for adjusting a sensor in the z-axis; FIG. 5(b) shows an actuator-controlled mechanism for adjusting a sensor in the z-axis; and (iii) FIG. 5(c) shows a rotatory mechanism for adjusting a sensor in the z-axis rotatory mechanism.

With reference to FIG. 5(a), the push button mechanism is effected by a multiple-membered mechanism comprising—(i) a retractable push button where the sensor is attached to, and (ii) a housing for sensor protection. In an inactivated state, the sensor is safely secured within the housing. In an activated state, i.e. the push button is pressed downwards towards a user's skin surface, the sensor is revealed out of the housing into various levels of contact pressure with the skin. This push button can be activated to achieve one and/or several fixed distances of protrusion, through mechanisms including but not limited to suction creation (such as a syringe plunger stopper); engagement of springs, ratchets and/or rotationally symmetrical barrels (such as in a retractable pen).

With reference to FIG. 5(b), the actuator-controlled mechanism is effected by a two-membered mechanism comprising a moving member attached to the sensor, and a stationary housing for sensor protection. In an inactivated state, the sensor is safely secured within the housing. In an activated state, the sensor is revealed into various levels of contact pressure with the skin.

With reference to FIG. 5(c), the rotatory mechanism is effected by a three-membered mechanism comprising a moving member attached to the sensor, a rotatory dial with mated screw grooves concentric to the moving member, and a housing to protect the sensor. Through a screw thread at the circumference this moving member, when the rotatory dial is effected in either clockwise and/or anti-clockwise movements, it translates rotational action (in the x- and y-axis) into z-axis movement downwards and upwards respectively.

In the abovementioned embodiments, the circumferential edges of the housing have an extended surface to effect an opposing force as a support to against the downward z-axis movement of the sensor. This surface serves as point of attachment for a securing/stabilizing means (e.g. a strap), where the extended surface can be in different configurations including but not limited to winged holders, attached rings, strap holes drilled at the rims.

### User-Controlled Positioning Features

With reference back to FIGS. 2(a), (b) and (c), in order to provide an indication or verification of an optimal position of the sensor/auscultation detector, a feedback mechanism can be provided, including:
a. Sound amplification through speakers 207. As the sensor 205 or detector module 202 is shifted across the arm, the volume varies based on an angle of placement. This guides a user on the most optimal placement over their vascular access;
b. Vascular access alignment indication 208 is a dial which perpendicularly aligns with the vascular access. Users can use this as a visual indicator to indicate the orientation of the sensor directly over the longitudinal axis of the vascular access; and/or
c. LED array 209 can provide signal strength indication. The acquisition of signals into the user module 104 may only be initiated if this LED array is completely lit.

### Auscultation Detector(s) Calibration for Signal Verification

As a means to ensure sensor functionality, an external device such as a detector calibration device and/or functional test device may be provided. Such devices can be used during a usable life of a detector module 402. The functional test device aids a user in verifying the accuracy of the stenosis sensor, e.g. by emitting a known signal for the stenosis sensor. In the event that the signal collection from the detector module 402 is beyond a pre-determined threshold, collection of signals from the vasculature by the detector module 402 is aborted.

In summary, to facilitate positioning of detectors for optimal signal acquisition, the following features are provided: (i) aligning/stabilizing features (e.g strap or sleeve); (ii) refinement of signals for fine-tuning via sound and visual feedback; and (iii) signal identification/verification and indicator to prompt user to begin signal acquisition.

Turning back to FIG. 1, the user module 104 is configured to provide communication between the detector module 102 and the processor module 106. The user module 104 can be configured to: (i) provide a first layer of computation capability for the collected signals from the detector module 102; (ii) establish communication to and from the processor module 106; and (iii) provides a user interface for a user to operate the system 100.

The user module 104 may include four sub-modules: (i) user interface 104a, (ii) microprocessor 104b, (iii) power supply 104c; and (iv) data storage 104d.

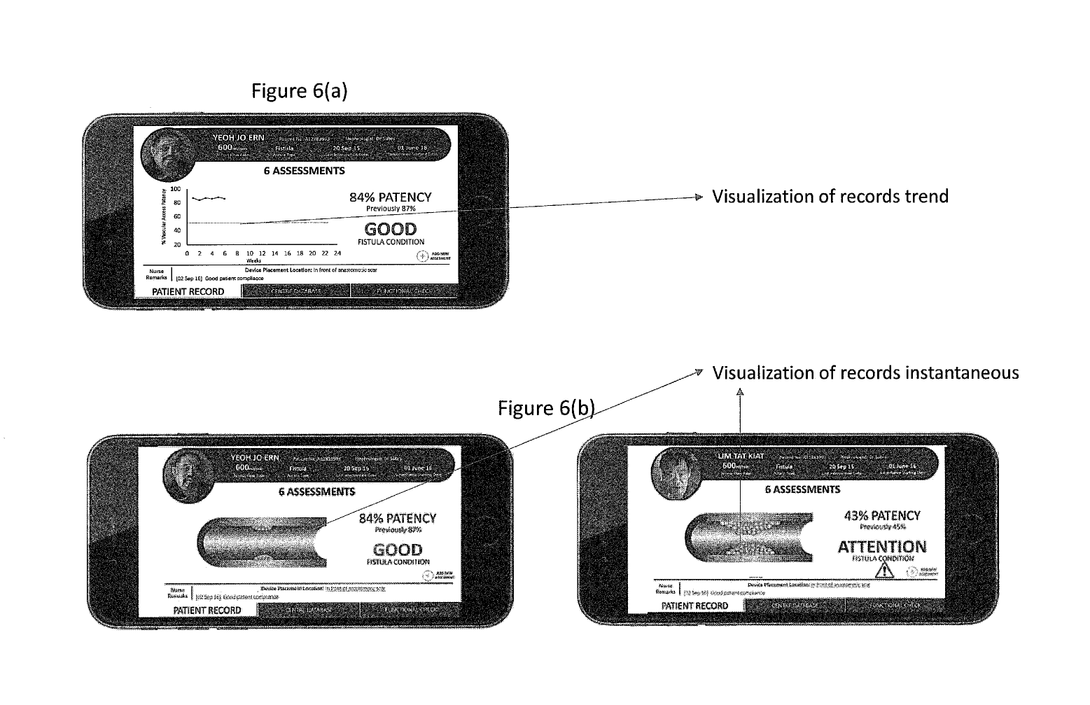

With reference to FIGS. 6(a) and (b), the user interface 104a comprises an interactive touch screen that displays a graphical user interface (GUI) that serves the following functions, but not limited to:
a. allow users to view individual user historical vascular access condition (see FIG. 6(a)), customisable summary compilation display, for example, summary overview of users from same dialysis session, same dialysis centre and/or same dialysis chains;
b. allow user to input user-specific information and comments when performing assessment;
c. activation of detector calibration and/or functional test; and
d. guides and/or prompts users on proper placement of detector module 402 through the use of visual and audio feedback.

This touch screen can be in the form of an electronic LED/LCD display, and implemented using other communication devices such as a mobile phone, smart phone, tablet, and Personal Digital Assistant (PDA).

In order to improve usability, other derivatives from vessel patency such as, but not limited to, visual/audio alert system for declining vascular access condition, stenosis prediction and comparative data from other similar demographic may also be displayed (see FIG. 6(b)).

Power supply 104c provides power for the user module 104, and can be in the form of e.g. dry cells or rechargeable dry cells. If a smart phone is used to implement the user module 104, the phone internal battery can be used to power the user module 104. The power supply 104c may also be used to power the detector module 102 in the case of a wired connection between the detector module 102 and the user module 104.

The internal data storage 104d within the user module 104 can be used to store critical user historical information for faster retrieval of information. The data storage 104d can also be used to store auscultation signals collected from the detector module 102 in the absence and/or unstable communication with the processor module 106 (if the processor module 106 is implemented as a remote module, e.g. using a cloud computing server). The locally stored auscultation signals can be transmitted to the processor module 106 after re-establishment of stable communication.

The microprocessor 104b functions as a micro-controller to control signal flow and time synchronise the auscultation signal collection from a detector module 102. The microprocessor 104b is also facilitates the following functions:
a. assess collected auscultation signal quality from each detector module;
b. guides a user on placement and z-axis of the detector module;
c. determines a distance between multiple detector modules (where applicable); and
d. performs functionality test during detector calibration and/or functional test.

In this case, the quality of the auscultation signal is a single or set of pre-defined quality parameters such as, but not limited to, detected pressure and/or auscultation signal strength.

The user module 104 is configured to perform a preliminary assessment of the collected auscultation signals to determine the quality of the collected signals. Based on this determination, users can be effectively guided on device placement. In this manner, active feedback can be provided to users. In contrast, prior art systems have sensors that are usually passive and only used for signal acquisition.

Turning back to FIG. 1, the processor module 106 can be implemented as a remote module, e.g. using a cloud computing server. The processor module 106 can be configured to:
a. receive auscultation signals from the user module 104;
b. process and analyse auscultation signals to identify acoustic signals indicative of vascular narrowing;
c. organize and store collected auscultation signals and all user information into a database; and
d. provide an indication of a user's vascular condition (present, e.g. as shown in FIG. 6(b); and historical information, e.g. as shown in FIG. 6(a)) to the user module 104 for display.

The processor module 106 may include two sub-modules: microprocessor 106a and data storage 106b. The data storage 106b comprises a customised database architecture that stores all user information. The microprocessor 106a controls data flow to and from the user module 104.

The processor module 106, with the microprocessor 106a and data storage 106b, is configured to execute a signal assessment algorithm. The signal assessment algorithm includes at least one of the following steps:
a. assess collected auscultation signal quality from the user module 104;
b. apply filtering techniques to the collected auscultation signal (e.g. for removal of background noise); and
c. identify acoustic signals indicative of vascular narrowing from the collected auscultation signal for assessment of vascular conditions.

In one exemplary embodiment, the signal processing involves extraction of pre-determined features that are directly and/or indirectly related to blood flow and/or indication of narrowing of a vascular lumen. These acoustic features indicative of vascular narrowing may include, but are not limited to, cardiac cycle window, signal amplitude, and energy spectrum.

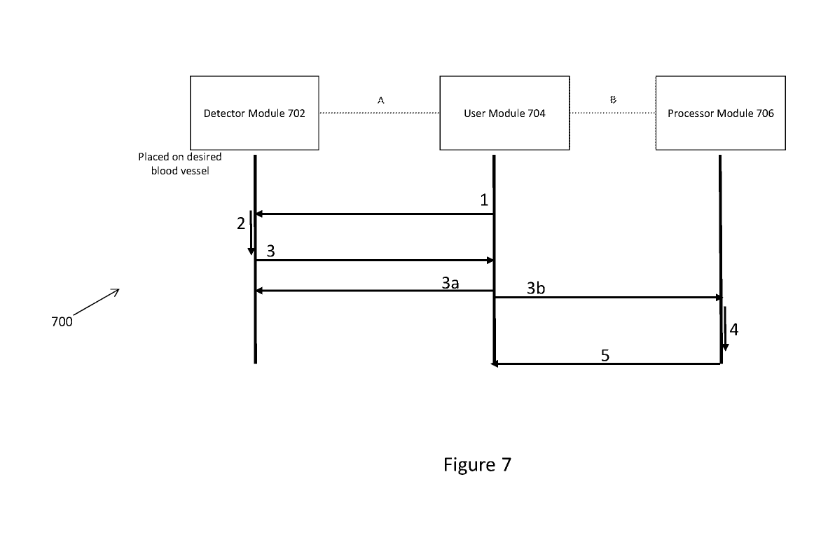

FIG. 7 shows a data flow diagram 700 illustrating a method for facilitating auscultation detection of vascular conditions, according to an example embodiment. At step 1, a user initiates an assessment session from a user module 704. Connection A is established to ensure detector module 702 is connected and ready. A quality check algorithm constantly ensures that acquired auscultation signals do not have any undesirable signals/artefacts (e.g. indicating sudden arm movements, speech functions, twitching fingers, etc.) by referencing a noise library.

At step 2, the detector module 702 (and its associated auscultation detectors) are positioned for optimal signal acquisition. This can be achieved through:
a. a stabilizing feature, e.g. a strap as described above in relation to FIGS. 2(a), (b) and (c); a sleeve as described above in relation to FIG. 3; and a cuff as described above in relation to FIGS. 4(a) and (b)(i)/(ii).
b. refinement of signals for fine-tuning through one or a combination of these methods: (i) audio feedback to a user by providing an indication of optimal sensor positioning (e.g. a continuous “beep” indicates optimal sensor position or an amplification of acoustic sounds from the blood vessel itself), (ii) visual feedback through matching the direction of vascular alignment indication, and (iii) visual feedback through LED lights indicating signal strength from vascular access. Multiple LED lights light up based on achievement of energy levels of pulsatile wave. This is to exclude sudden impact, taps, etc.
c. When positioned correctly, an optimized energy level threshold is achieved. The user is then allowed to initiate signal acquisition.
In the case of a plurality of detectors arranged in a 2D array, each of the detectors may return a reading and a signal quality (e.g. signal strength, signal to noise ratio, etc.) of each reading is determined. The readings from all detectors are compared and only the reading with the best signal quality (e.g. highest signal strength) is collected/acquired.

At step 3, a positioning and a quality check algorithm repeatedly checks acquired auscultation signals while signal data is transferred through communication A. If either the positioning or the quality check fails, new signals are required from the detector module 702 by returning back to step 2 for re-initiation of alignment. On the other hand, if both the positioning and the quality check pass, the auscultation signals are sent to a processor module 706 via Communication B for analysis.

At step 4, the processor module 706 processes the signals through filtering a desirable frequency range and analysis of acoustic features indicative of vascular narrowing to derive a degree of blockage of a blood vessel.

At step 5, the degree of blockage is stored and returned to the user module 704 for display.

According to an exemplary embodiment, there is provided a system for facilitating auscultation detection of vascular conditions, comprising: a detector module comprising one or more auscultation detectors, the detector module configured to acquire auscultation signals associated with at least one blood vessel; a user module in communication with the detector module; and a processor module in communication with the user module. The user module is configured to relay the auscultation signals from the detector module to the processor module. The processor module is configured to process the auscultation signals to generate corresponding blood flow characteristic data of the at least one blood vessel. The blood flow characteristic data indicative of one or more vascular conditions. The one or more vascular conditions may include an extent of blockage of a blood vessel and/or duration (length of time) before medical intervention is required.

The processor module may be configured to filter a pre-determined frequency range of the auscultation signals and analyse features within the filtered frequency range to identify acoustic features indicative of vascular narrowing.

The user module may comprise a memory module having stored therein an artefact library. Accordingly, the user module can be further configured to reference the artefact library to determine a presence of artefacts in the auscultation signals received from the detector module. The user module may be configured to transmit the auscultation signals to the processor module on a condition that the presence of artefacts in the auscultation signals received from the detector module is within a pre-determined threshold.

The detector module may be configured to transmit the auscultation signals to the user module on a condition that a position of the detector module and/or the one or more auscultation detectors with respect to at least one blood vessel is within a pre-determined threshold.

Embodiments of the invention seek to simplify blood vessel blockage assessment such that patients can conduct the assessment by themselves. Features such as sensory feedback (sounds, tactile feel, visuals through LEDs and user module, etc.) can facilitate patient-initiated assessment. Currently, only nurses or ultrasound technologists conduct blood vessel blockage assessment.

Furthermore, embodiments of the invention seek to address the inadequacy of current commercially available vascular condition assessment techniques at detecting early stage vascular complications, such as lengthy assessment duration and restriction to in-hospital/in-dialysis centre usage. For example, in the case of the assessment of the vascular access for hemodialysis, a commercially available device, Transonic®, involves a lengthy and manual assessment process that not only requires a multiple-step assessment process per assessment (including setup, calibration and assessment). This relatively long assessment duration is a significant deterrent and limits ease of achieving desired assessment regularity. In contrast, embodiments of the invention seek to provide relatively shorter assessment duration of about 1 minute or less.

The non-invasive assessment nature and the ease of usage (i.e. minimal skillset required for usage) of embodiments of the invention, means that renal nurses, care-givers and even the patients themselves can operate embodiments of the invention. Further, the non-invasive assessment nature of embodiments of the invention enable signal collection regardless of blood vessel anatomy, where embodiments exert a radial pressure distribution method to stabilize signal acquisition and positioning. In contrast, current techniques are typically involves passive needling, skin contact via hand placement, adhesives, etc.

Embodiments of the invention can also be customised for group use (for hospital units, dialysis centres and/or elderly care centres) or for individual use (patients can use embodiments as a home-based personal management tool). This removes all restrictions and limitations that users currently experience from the commercially available assessment techniques.

When used on a regular (time-separated) basis, embodiments of the invention can generate a prospective trend and assessment of vascular conditions. Embodiments of the invention enable frequent assessment (even several times daily) and this is crucial to addressing the inadequacy of current techniques at detecting early stage complications.

In summary, embodiments of the invention seek to provide systems and methods for facilitating auscultation detection of vascular conditions that are highly portable, skillset independent and location independent.

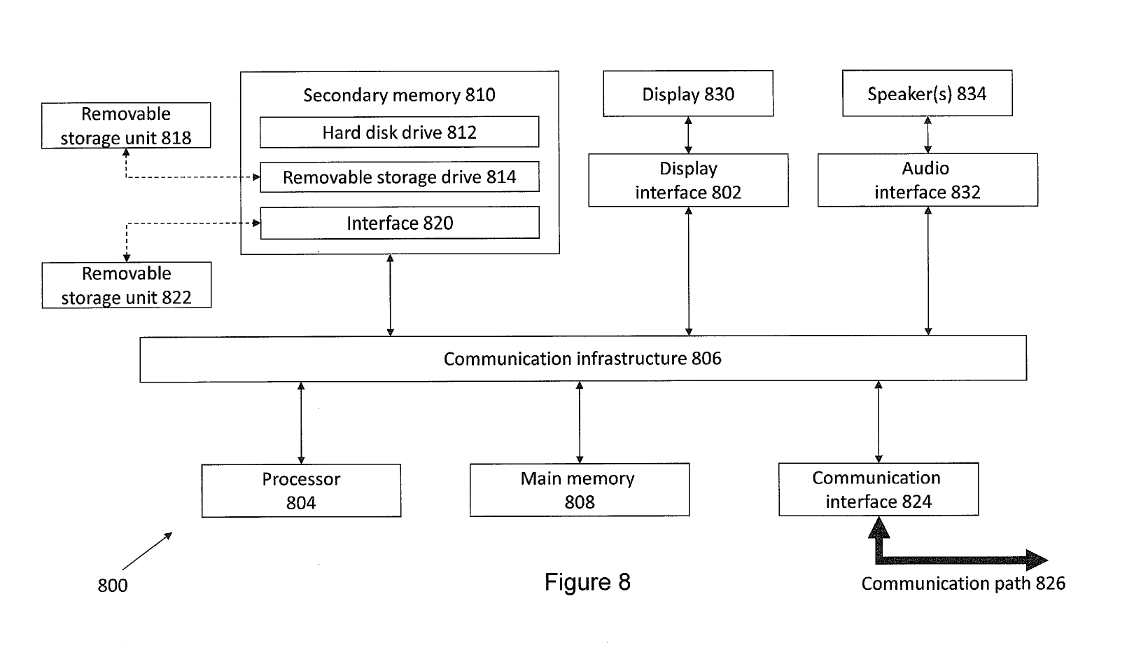

FIG. 8 shows a schematic diagram of a computer system 800 suitable for use in executing at least some steps of the method for facilitating auscultation detection of vascular conditions and/or for realizing at least a part of the system for facilitating auscultation detection of vascular conditions (e.g. the user module 104 or processor module 106).

The following description of the computer system/computing device 800 is provided by way of example only and is not intended to be limiting.

As shown in FIG. 8, the example computing device 800 includes a processor 804 for executing software routines. Although a single processor is shown for the sake of clarity, the computing device 800 may also include a multi-processor system. The processor 804 is connected to a communication infrastructure 806 for communication with other components of the computing device 800. The communication infrastructure 806 may include, for example, a communications bus, cross-bar, or network.

The computing device 800 further includes a main memory 808, such as a random access memory (RAM), and a secondary memory 810. The secondary memory 810 may include, for example, a hard disk drive 812 and/or a removable storage drive 814, which may include a magnetic tape drive, an optical disk drive, or the like. The removable storage drive 814 reads from and/or writes to a removable storage unit 818 in a well-known manner. The removable storage unit 818 may include a magnetic tape, optical disk, or the like, which is read by and written to by removable storage drive 814. As will be appreciated by persons skilled in the relevant art(s), the removable storage unit 818 includes a computer readable storage medium having stored therein computer executable program code instructions and/or data.

In an alternative implementation, the secondary memory 810 may additionally or alternatively include other similar means for allowing computer programs or other instructions to be loaded into the computing device 800. Such means can include, for example, a removable storage unit 822 and an interface 820. Examples of a removable storage unit 822 and interface 820 include a removable memory chip (such as an EPROM or PROM) and associated socket, and other removable storage units 822 and interfaces 820 which allow software and data to be transferred from the removable storage unit 822 to the computer system 800.

The computing device 800 also includes at least one communication interface 824. The communication interface 824 allows software and data to be transferred between computing device 800 and external devices via a communication path 826. In various embodiments, the communication interface 824 permits data to be transferred between the computing device 800 and a data communication network, such as a public data or private data communication network. The communication interface 824 may be used to exchange data between different computing devices 800 which such computing devices 800 form part an interconnected computer network. Examples of a communication interface 824 can include a modem, a network interface (such as an Ethernet card), a communication port, an antenna with associated circuitry and the like. The communication interface 824 may be wired or may be wireless. Software and data transferred via the communication interface 824 are in the form of signals which can be electronic, electromagnetic, optical or other signals capable of being received by communication interface 824. These signals are provided to the communication interface via the communication path 826.

Optionally, the computing device 800 further includes a display interface 802 which performs operations for rendering images to an associated display 830 and an audio interface 832 for performing operations for playing audio content via associated speaker(s) 834.

As used herein, the term “computer program product” may refer, in part, to removable storage unit 818, removable storage unit 822, a hard disk installed in hard disk drive 812, or a carrier wave carrying software over communication path 826 (wireless link or cable) to communication interface 824. Computer readable storage media refers to any non-transitory tangible storage medium that provides recorded instructions and/or data to the computing device 800 for execution and/or processing. Examples of such storage media include floppy disks, magnetic tape, CD-ROM, DVD, Blu-ray™ Disc, a hard disk drive, a ROM or integrated circuit, USB memory, a magneto-optical disk, or a computer readable card such as a PCMCIA card and the like, whether or not such devices are internal or external of the computing device 800. Examples of transitory or non-tangible computer readable transmission media that may also participate in the provision of software, application programs, instructions and/or data to the computing device 800 include radio or infra-red transmission channels as well as a network connection to another computer or networked device, and the Internet or Intranets including e-mail transmissions and information recorded on Websites and the like.

The computer programs (also called computer program code) are stored in main memory 808 and/or secondary memory 810. Computer programs can also be received via the communication interface 824. Such computer programs, when executed, enable the computing device 800 to perform one or more features of embodiments discussed herein. In various embodiments, the computer programs, when executed, enable the processor 804 to perform features of the above-described embodiments. Accordingly, such computer programs represent controllers of the computer system 800.

Software may be stored in a computer program product and loaded into the computing device 800 using the removable storage drive 814, the hard disk drive 812, or the interface 820. Alternatively, the computer program product may be downloaded to the computer system 800 over the communications path 826. The software, when executed by the processor 804, causes the computing device 800 to perform functions of embodiments described herein.

It is to be understood that the embodiment of FIG. 8 is presented merely by way of example. Therefore, in some embodiments one or more features of the computing device 800 may be omitted. Also, in some embodiments, one or more features of the computing device 800 may be combined together. Additionally, in some embodiments, one or more features of the computing device 800 may be split into one or more component parts.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1. A detector module for facilitating auscultation detection of vascular conditions, comprising:
one or more auscultation detectors, each of the one or more auscultation detectors configured to acquire auscultation signals associated with at least one blood vessel;
an attacher which is configured to secure the detector module superficially onto a user's skin such that the auscultation signals associated with the at least one blood vessel can be acquired; and
a microprocessor module that is in communication with the one or more auscultation detectors and is configured to transmit the acquired auscultation signals to an external module that is in communication with the detector module.

2. The detector module as claimed in claim 1, comprising a plurality of the auscultation detectors, wherein the microprocessor module is further configured to:
receive acquired auscultation signals from the plurality of auscultation detectors; and
determine which acquired auscultation signals to transmit to the external module based on one or more pre-defined parameters.

3. The detector module as claimed in claim 2, wherein the one or more pre-defined parameters comprises:
detected pressure of the auscultation detector(s) against the user's skin;
signal-to-noise ratio;
dynamic range of the auscultation detector(s);
frequency response of the auscultation detector(s); and/or
auscultation signal strength.

4. The detector module as claimed in claim 3, wherein the acquired auscultation signal with a highest auscultation signal strength is transmitted to the external module.

5. The detector module as claimed in claim 1, wherein each of the one or more auscultation detectors comprises an actuating mechanism that is configured to move the auscultation detector along a z-axis, either away or towards the user's skin.

6. The detector module as claimed in claim 5, wherein the microprocessor module is further configured to:
determine a distance between the auscultation detector(s) and the user's skin or the at least one blood vessel; and
provide a feedback to the user that is indicative of the distance between the auscultation detector(s) and the user's skin or the at least one blood vessel.

7. The detector module as claimed in claim 1, wherein the microprocessor module is further configured to process the acquired auscultation signals to generate corresponding blood flow characteristic data of the at least one blood vessel, wherein the blood flow characteristic data comprises acoustic signals indicative of vascular narrowing.

8. The detector module as claimed in claim 1, further comprising a memory module having stored therein an artefact library, and wherein the microprocessor module is further configured to reference the artefact library to determine a presence of artefacts in the auscultation signals associated with the at least one blood vessel acquired by the one or more auscultation detectors.

9. A method for facilitating auscultation detection of vascular conditions, comprising:
providing a detector module comprising one or more auscultation detectors, each of the one or more auscultation detectors configured to acquire auscultation signals associated with at least one blood vessel;
providing an attacher which is configured to secure the detector module superficially onto a user's skin such that the auscultation signals associated with the at least one blood vessel can be acquired; and
providing a microprocessor module that is in communication with the detector module, wherein the microprocessor module is configured to transmit the acquired auscultation signals to an external module that is in communication with the detector module.

10. The method as claimed in claim 9, further comprising:
providing a plurality of the auscultation detectors; and
configuring the microprocessor module to:
receive acquired auscultation signals from the plurality of auscultation detectors, and
determine which acquired auscultation signals to transmit to the external module based on one or more pre-defined parameters.

11. The method as claimed in claim 10, wherein the one or more pre-defined parameters comprises:
detected pressure of the auscultation detector(s) against the user's skin;
signal-to-noise ratio;
dynamic range of the auscultation detector(s);
frequency response of the auscultation detector(s); and/or
auscultation signal strength.

12. The method as claimed in claim 11, wherein the acquired auscultation signal with a highest auscultation signal strength is transmitted to the external module.

13. The method as claimed in claim 9, further comprising:
providing each of the one or more auscultation detectors with an actuating mechanism that is configured to move the auscultation detector along a z-axis, either away or towards the user's skin.

14. The method as claimed in claim 13, further comprising:
configuring the microprocessor module to:
determine a distance between the auscultation detector(s) and the user's skin or the at least one blood vessel, and
provide a feedback to the user that is indicative of the distance between the auscultation detector(s) and the user's skin or the at least one blood vessel.

15. The method as claimed in claim 9, further comprising:
configuring the microprocessor module to:
process the acquired auscultation signals to generate corresponding blood flow characteristic data of the at least one blood vessel, wherein the blood flow characteristic data comprises acoustic signals indicative of vascular narrowing.

16. The method as claimed in claim 9, further comprising:
providing a memory module having stored therein an artefact library, the memory module being in communication with the microprocessor module; and
configuring the microprocessor module to:
reference the artefact library to determine a presence of artefacts in the auscultation signals associated with the at least one blood vessel acquired by the one or more auscultation detectors.

17. A detector module for detecting vascular conditions, the detector comprising:
auscultation detectors which are configured to acquire auscultation signals associated with a blood vessel;
an attacher which secures the detector module superficially onto a user's skin to acquire auscultation signals associated with the blood vessel; and
a processor module which is configured to receive the auscultation signals sensed by the auscultation detections, the processor module processes the auscultation signals to determine which auscultation signals to transmit to an external module which is external to the detector module with the processor module, wherein determining which auscultation signals to transmit to the external module is based on one or more pre-defined parameters, wherein the one or more pre-defined parameters comprises:
detected pressure of the auscultation detectors against the user's skin,
signal-to-noise ratio,
dynamic range of the auscultation detectors,
frequency response of the auscultation detectors, and/or
auscultation signal strength.

18. The detector module of claim 17, wherein the acquired auscultation signal with a highest auscultation signal strength is transmitted to the external module.

19. The detector module of claim 17, wherein the auscultation detectors comprise an actuating mechanism that is configured to move the auscultation detectors along a z-axis, either away or towards the user's skin.

20. The detector module of claim 16, wherein the processor module is configured to process the acquired auscultation signals to generate corresponding blood flow characteristic data of the blood vessel, wherein the blood flow characteristic data comprises acoustic signals indicative of vascular narrowing.
